(19) United States
(12) Patent Application Publication
(10) Pub. No.: US 2025/0152412 A1
(43) Pub. Date: May 15, 2025

(54) DEVICES, APPLICATION SYSTEMS AND METHODS WITH LOCALIZED HEAT FLUX ZONES FOR REMOVING HEAT FROM SUBCUTANEOUS LIPID-RICH CELLS

(71) Applicant: Zeltiq Aesthetics, Inc., North Chicago, IL (US)

(72) Inventors: Bryan Weber, Livermore, CA (US); Joseph Coakley, Dublin, CA (US); John Potosky, San Jose, CA (US)

(21) Appl. No.: 18/892,261

(22) Filed: Sep. 20, 2024

Related U.S. Application Data
(63) Continuation of application No. 16/905,889, filed on Jun. 18, 2020, now abandoned.
Application No. 16/905,889 is a continuation of application No. 13/358,337, filed on Jan. 25, 2012, now Pat. No. 10,722,395.
(60) Provisional application No. 61/435,944, filed on Jan. 25, 2011.

(51) Int. Cl. A61F 7/10; A61B 90/00; A61F 7/00; A61F 7/02

(52) U.S. Cl. CPC A61F 7/10; A61F 7/02; A61B 2090/0463; A61F 2007/0056; A61F 2007/0075; A61F 2007/0095; A61F 2007/029

(57) ABSTRACT

Application systems, disposable interface assemblies and methods for cooling subcutaneous lipid-rich tissue. One embodiment of an application system includes a cooling unit, a cryoprotectant vessel, a contact member and an array of selectively addressable heating elements. The cryoprotectant vessel is configured to contain a fluidic cryoprotectant such that at least a portion of the cryoprotectant is cooled by the cooling unit to a desired base temperature. The contact member is attached to the cryoprotectant vessel and includes a backside in contact with the cryoprotectant and a front side opposite the backside. The contact member is configured to allow the cryoprotectant to flow from the backside to the front side. The array of selectively addressable heating elements is carried by the contact member.

### CROSS-REFERENCE TO RELATED APPLICATION(S)

This application claims priority to U.S. Provisional Patent Application No. 61/435,944, filed Jan. 25, 2011, entitled “DEVICES, APPLICATION SYSTEMS AND METHODS WITH LOCALIZED HEAT FLUX ZONES FOR REMOVING HEAT FROM SUBCUTANEOUS LIPID-RICH CELLS,” which is incorporated herein by reference in its entirety.

The following commonly-assigned U.S. patent applications are incorporated herein by reference in their entirety:
U.S. Patent Publication No. 2008/0287839 entitled “METHOD OF ENHANCED REMOVAL OF HEAT FROM SUBCUTANEOUS LIPID-RICH CELLS AND TREATMENT APPARATUS HAVING AN ACTUATOR”;
U.S. Pat. No. 6,032,675 entitled “FREEZING METHOD FOR CONTROLLED REMOVAL OF FATTY TISSUE BY LIPOSUCTION”;
U.S. Patent Publication No. 2007/0255362 entitled “CRYOPROTECTANT FOR USE WITH A TREATMENT DEVICE FOR IMPROVED COOLING OF SUBCUTANEOUS LIPID-RICH CELLS”;
U.S. Pat. No. 7,854,754 entitled “COOLING DEVICE FOR REMOVING HEAT FROM SUBCUTANEOUS LIPID-RICH CELLS”;
U.S. Patent Publication No. 2011/0066216 entitled “COOLING DEVICE FOR REMOVING HEAT FROM SUBCUTANEOUS LIPID-RICH CELLS”;
U.S. Patent Publication No. 2008/0077201 entitled “COOLING DEVICES WITH FLEXIBLE SENSORS”;
U.S. Patent Publication No. 2008/0077211 entitled “COOLING DEVICE HAVING A PLURALITY OF CONTROLLABLE COOLING ELEMENTS TO PROVIDE A PREDETERMINED COOLING PROFILE”;
U.S. Patent Publication No. 2009/0118722, filed Oct. 31, 2007, entitled “METHOD AND APPARATUS FOR COOLING SUBCUTANEOUS LIPID-RICH CELLS OR TISSUE”;
U.S. Patent Publication No. 2009/0018624 entitled “LIMITING USE OF DISPOSABLE SUBJECT 11 PROTECTION DEVICES”;
U.S. Patent Publication No. 2009/0018623 entitled “SYSTEM FOR TREATING LIPID-RICH REGIONS”;
U.S. Patent Publication No. 2009/0018625 entitled “MANAGING SYSTEM TEMPERATURE TO REMOVE HEAT FROM LIPID-RICH REGIONS”;
U.S. Patent Publication No. 2009/0018627 entitled “SECURE SYSTEM FOR REMOVING HEAT FROM LIPID-RICH REGIONS”;
U.S. Patent Publication No. 2009/0018626 entitled “USER INTERFACES FOR A SYSTEM THAT REMOVES HEAT FROM LIPID-RICH REGIONS”;
U.S. Pat. No. 6,041,787 entitled “USE OF CRYOPROTECTIVE AGENT COMPOUNDS DURING CRYOSURGERY”;
U.S. Patent Publication No. 2009/0149929 entitled “MONITORING THE COOLING OF SUBCUTANEOUS LIPID-RICH CELLS, SUCH AS THE COOLING OF ADIPOSE TISSUE”;
U.S. Provisional Patent Application Ser. No. 60/941,567 entitled “METHODS, APPARATUSES AND SYSTEMS FOR COOLING THE SKIN AND SUBCUTANEOUS TISSUE”;
U.S. Patent Publication No. 2010/0081971 entitled “TREATMENT PLANNING SYSTEMS AND METHODS FOR BODY CONTOURING APPLICATIONS”;
U.S. patent application Ser. No. 12/275,002 entitled “APPARATUS WITH HYDROPHILIC RESERVOIRS FOR COOLING SUBCUTANEOUS LIPID-RICH CELLS”;
U.S. patent application Ser. No. 12/275,014 entitled “APPARATUS WITH HYDROPHOBIC FILTERS FOR REMOVING HEAT FROM SUBCUTANEOUS LIPID-RICH CELLS”;
U.S. Patent Publication No. 2010/0152824 entitled “SYSTEMS AND METHODS WITH INTERRUPT/RESUME CAPABILITIES FOR COOLING SUBCUTANEOUS LIPID-RICH CELLS”;
U.S. Patent Publication No. 2008/0077202 entitled “TISSUE TREATMENT METHODS”;
U.S. Patent Publication No. 2010/0280582 entitled “DEVICE, SYSTEM AND METHOD FOR REMOVING HEAT FROM SUBCUTANEOUS LIPID-RICH CELLS”;
U.S. patent application Ser. No. 12/840,235 entitled “COMBINED MODALITY TREATMENT SYSTEMS, METHODS AND APPARATUS FOR BODY CONTOURING APPLICATIONS”;
U.S. Publication No. 2011/0238050 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”; and
U.S. Publication No. 2011/0238051 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”.

### TECHNICAL FIELD

The present application relates generally to devices, application systems and methods for removing heat from subcutaneous lipid-rich cells. In particular, several embodiments are directed toward a device that provides independent control of the heat flux through a plurality of zones based on a desired heat flux profile.

### BACKGROUND

Excess body fat, or adipose tissue, may be present in various locations of the body, including, for example, the thigh, buttocks, abdomen, knees, back, face, arms, chin, and other areas. Moreover, excess adipose tissue is thought to magnify the unattractive appearance of cellulite, which forms when subcutaneous fat protrudes into the dermis and creates dimples where the skin is attached to underlying structural fibrous strands. Cellulite and excessive amounts of adipose tissue are often considered to be unappealing. Moreover, significant health risks may be associated with higher amounts of excess body fat.

A variety of methods have been used to treat individuals having excess body fat and, in many instances, non-invasive removal of excess subcutaneous adipose tissue can eliminate unnecessary recovery time and discomfort associated with invasive procedures such as liposuction. Conventional non-invasive treatments for removing excess body fat typically include topical agents, weight-loss drugs, regular exercise, dieting or a combination of these treatments. One drawback of these treatments is that they may not be effective or even possible under certain circumstances. For example, when a person is physically injured or ill, regular exercise may not be an option. Similarly, weight-loss drugs or topical agents are not an option when they cause an allergic or negative reaction. Furthermore, fat loss in selective areas of a person's body often cannot be achieved using general or systemic weight-loss methods.

Other methods designed to reduce subcutaneous adipose tissue include laser-assisted liposuction and mesotherapy. Newer non-invasive methods include applying radiant energy to subcutaneous lipid-rich cells via, e.g., radio frequency and/or light energy, such as described in U.S. Patent Publication No. 2006/0036300 and U.S. Pat. No. 5,143,063, or via, e.g., high intensity focused ultrasound (HIFU) radiation such as described in U.S. Pat. Nos. 7,258,674 and 7,347,855. In contrast, methods and devices for non-invasively reducing subcutaneous adipose tissue by cooling are disclosed in U.S. Pat. No. 7,367,341 entitled “METHODS AND DEVICES FOR SELECTIVE DISRUPTION OF FATTY TISSUE BY CONTROLLED COOLING” to Anderson et al. and U.S. Patent Publication No. 2005/0251120 entitled “METHODS AND DEVICES FOR DETECTION AND CONTROL OF SELECTIVE DISRUPTION OF FATTY TISSUE BY CONTROLLED COOLING” to Anderson et al., the entire disclosures of which are incorporated herein by reference.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not necessarily drawn to scale, and some of these elements are arbitrarily enlarged and positioned to improve drawing legibility. Further, the particular shapes of the elements as drawn are not necessarily intended to convey any information regarding the actual shape of the particular elements, and are generally selected for ease of recognition in the drawings.

FIG. 1 is an isometric view schematically illustrating a treatment system for treating subcutaneous lipid-rich regions of a subject in accordance with an embodiment of the technology.

FIG. 2A is a schematic cross-sectional view of an application system for cooling subcutaneous lipid-rich tissue in accordance with an embodiment of the technology.

FIG. 2B is a schematic bottom view of an interface assembly of an application system for cooling subcutaneous lipid-rich tissue in accordance with an embodiment of the technology.

FIG. 3 is a schematic cross-sectional view of an application system for cooling subcutaneous lipid-rich tissue in accordance with an embodiment of the technology.

FIG. 4 is a schematic cross-sectional view of an interface element of an interface assembly for cooling subcutaneous lipid-rich tissue in accordance with an embodiment of the technology.

FIGS. 5A and 5B are schematic views of an implementation of an application system for cooling subcutaneous lipid-rich tissue in accordance with an embodiment of the technology.

FIG. 6 is a schematic cross-sectional view of an application system for cooling subcutaneous lipid-rich tissue in accordance with an embodiment of the technology.

FIG. 7 is a schematic cross-sectional view of a treatment device with an application system for cooling subcutaneous lipid-rich tissue in accordance with an embodiment of the technology.

### DETAILED DESCRIPTION

### Overview

Several examples of devices, application systems and methods for independently controlling the heat flux through a plurality of cooling zones for cooling subcutaneous adipose tissue in accordance with the technology are described below. Although the following description provides many specific details of the following examples in a manner sufficient to enable a person skilled in the relevant art to practice, make and use them, several of the details and advantages described below may not be necessary to practice certain examples and methods of the technology. Additionally, the technology may include other examples and methods that are within the scope of the claims but are not described in detail.

Reference throughout this specification to “one example,” “an example,” “one embodiment” or “an embodiment” means that a particular feature, structure, or characteristic described in connection with the example is included in at least one example of the present technology. Thus, the occurrences of the phrases “in one example,” “in an example,” “one embodiment” or “an embodiment” in various places throughout this specification are not necessarily all referring to the same example. Furthermore, the particular features, structures, routines, steps or characteristics may be combined in any suitable manner in one or more examples of the technology. The headings provided herein are for convenience only and are not intended to limit or interpret the scope or meaning of the claimed technology.

One embodiment of an application system for cooling subcutaneous lipid-rich tissue comprises a cooling unit, a cryoprotectant vessel, a contact member and an array of selectably addressable heating elements. The cryoprotectant vessel is configured to contain a fluidic cryoprotectant such that at least a portion of the cryoprotectant is cooled by the cooling unit to a desired base temperature. The contact member is attached to the cryoprotectant vessel, and the contact member includes a backside in contact with the cryoprotectant and a front side opposite the backside. The contact member is configured to allow the cryoprotectant to flow from the backside to the front side, and the array of selectably addressable heating elements is carried by the contact member.

One embodiment of a method of operating an application system for cooling subcutaneous lipid-rich tissue includes cooling a cryoprotectant to a base temperature below 37° C. The method further includes passing the cyroprotectant through a flexible contact member of an interface element, and selectively heating at least one heating element of an array of heating elements carried by the flexible contact member to a temperature different than other heating elements of the array. As a result, the temperature of the cryoprotectant proximate to the heated heating element is raised to a contact temperature higher than the base temperature.

In a more detailed embodiment of an application system for cooling subcutaneous lipid-rich tissue, the cooling unit comprises a heat exchanger having a coolant chamber through which a coolant can flow. The cryoprotectant vessel comprises a back panel and a sidewall projecting from the back panel. The contact member is a flexible barrier attached to the sidewall of the cryoprotectant vessel to form a cryoprotectant chamber, and the flexible barrier and the cryoprotectant vessel together form a disposable interface element. The flexible barrier, for example, can be a porous membrane or other flexible panel with small holes. The interface assembly further comprises a connector that couples the cryoprotectant vessel to the cooling unit. The application system of this embodiment further comprises an array of temperature sensor sets carried by the flexible membrane, and each individual heating element is associated with a corresponding temperature sensor set. Additionally, the application system can optionally comprise a larger heating element spaced apart from the backside of the array of temperature sensor sets and a controller. The controller includes a computer-operable medium programmed to receive sensed temperatures from the temperature sensor sets and adjust the associated heating elements based on the sensed temperatures and a desired heating profile to thereby provide localized temperature differentials in the cryoprotectant corresponding to the desired heating profile.

### General System Components

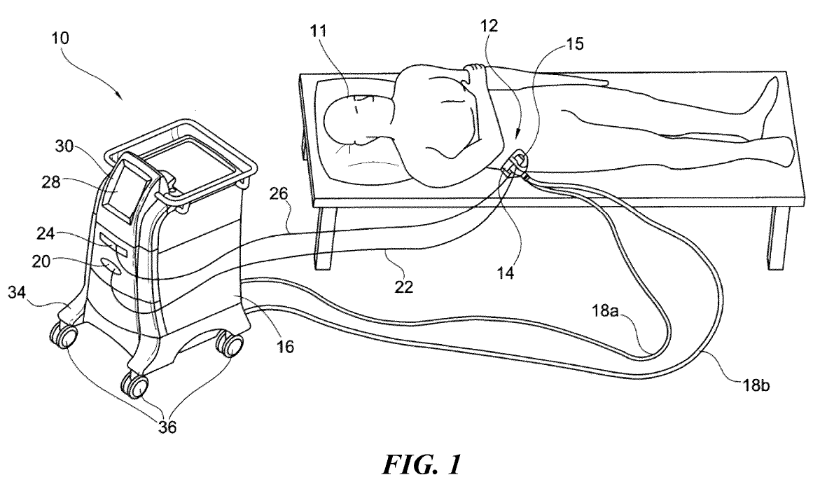

FIG. 1 and the following discussion provide a brief, general description of a suitable treatment system 10 in which aspects of the present technology can be implemented. Those skilled in the relevant art will appreciate that the present technology can be practiced with other systems and treatment protocols, including invasive, minimally invasive, other non-invasive cosmetic or medical treatment systems and/or combinations of one or more of the above for treating a subject 11. In general, the term “treatment system”, as used generally herein, refers to any of the above system categories of cosmetic or medical treatments as well as any treatment regimes or medical device usage.

The treatment system 10 is suitable for cooling the subcutaneous adipose tissue of a subject 11 in a manner that reduces the volume of the adipose tissue. “Subcutaneous tissue” can include tissue lying beneath the dermis and includes subcutaneous fat, or adipose tissue that may be composed primarily of lipid-rich cells, or adipocytes. When cooling subcutaneous tissues to a temperature lower than 37° C., subcutaneous lipid-rich cells can be affected selectively. In general, the epidermis and dermis of the subject 11 lack lipid-rich cells compared to the underlying lipid-rich cells forming the adipose tissue. Because non-lipid-rich cells usually can withstand colder temperatures better than lipid-rich cells, the subcutaneous lipid-rich cells can be affected selectively without affecting the non-lipid-rich cells in the dermis, epidermis and other surrounding tissue. In some embodiments, the treatment system 100 can apply cooling temperatures to the skin of the subject 11 in a range of about −20° C. to about 20° C. In other embodiments, the cooling temperatures can be from about −20° C. to about 10° C., approximately 0° C. to approximately 20° C., about −15° C. to about 5° C., approximately −5° C. to approximately 15° C., or about −10° C. to about 0° C.

Without being bound by theory, the selective effect of cooling on lipid-rich cells is believed to result in, for example, membrane disruption, cell shrinkage, disabling, destroying, removing, killing or other methods of lipid-rich cell alteration. Such alteration is believed to stem from one or more mechanisms acting alone or in combination. It is thought that such mechanism(s) trigger an apoptotic cascade, which is believed to be the dominant form of lipid-rich cell death by non-invasive cooling.

Apoptosis, also referred to as “programmed cell death”, is a genetically-induced death mechanism by which cells self-destruct without incurring damage to surrounding tissues. An ordered series of biochemical events induce cells to morphologically change. These changes include cellular blebbing, loss of cell membrane asymmetry and attachment, cell shrinkage, chromatin condensation and chromosomal DNA fragmentation. Injury via an external stimulus, such as cold exposure, is one mechanism that can induce cellular apoptosis in cells. Nagle, W. A., Soloff, B. L., Moss, A. J. Jr., Henle, K. J. “Cultured Chinese Hamster Cells Undergo Apoptosis After Exposure to Cold but Nonfreezing Temperatures” Cryobiology 27, 439-451 (1990).

One aspect of apoptosis, in contrast to cellular necrosis (a traumatic form of cell death causing local inflammation), is that apoptotic cells express and display phagocytic markers on the surface of the cell membrane, thus marking the cells for phagocytosis by macrophages. As a result, phagocytes can engulf and remove the dying cells (e.g., the lipid-rich cells) without eliciting an immune response. Temperatures that elicit these apoptotic events in lipid-rich cells may contribute to long-lasting and/or permanent reduction and reshaping of subcutaneous adipose tissue.

One mechanism of apoptotic lipid-rich cell death by cooling is believed to involve localized crystallization of lipids within the adipocytes at temperatures that do not induce crystallization in non-lipid-rich cells. The crystallized lipids selectively may injure these cells, inducing apoptosis (and may also induce necrotic death if the crystallized lipids damage or rupture the bi-lipid membrane of the adipocyte). Another mechanism of injury involves the lipid phase transition of those lipids within the cell's bi-lipid membrane, which results in membrane disruption, thereby inducing apoptosis. This mechanism is well-documented for many cell types and may be active when adipocytes, or lipid-rich cells, are cooled. Mazur, P., “Cryobiology: the Freezing of Biological Systems” Science, 68:939-949 (1970); Quinn, P. J., “A Lipid Phase Separation Model of Low Temperature Damage to Biological Membranes” Cryobiology, 22:128-147 (1985); Rubinsky, B., “Principles of Low Temperature Preservation” Heart Failure Reviews, 8, 277-284 (2003). Other yet-to-be understood apoptotic mechanisms may exist, based on the relative sensitivity of lipid-rich cells to cooling compared to non-lipid rich cells.

In addition to the apoptotic mechanisms involved in lipid-rich cell death, local cold exposure also is believed to induce lipolysis (i.e., fat metabolism) of lipid-rich cells and has been shown to enhance existing lipolysis which serves to further increase the reduction in subcutaneous lipid-rich cells. Vallerand, A. L., Zamecnik. J., Jones, P. J. H., Jacobs, I. “Cold Stress Increases Lipolysis, FFA Ra and TG/FFA Cycling in Humans” Aviation, Space and Environmental Medicine 70, 42-50 (1999).

In various embodiments, the treatment system 10 includes a controller, a computing device, a data acquisition device, a chiller, and one or more treatment devices. These components can be implemented in various embodiments to apply selected treatment profiles to a subject 11 (e.g., a human or animal) for reducing adipose tissue.

FIG. 1 is a perspective view illustrating one example of a treatment system 10 for non-invasively removing heat from subcutaneous lipid-rich target areas of the subject 11, such as an abdominal area 12 or another suitable area. The system 10 may include a treatment device 14 that engages the target area of the subject 11 and a treatment unit 16 that operate together to cool or otherwise remove heat from the subcutaneous lipid-rich cells of the subject 11. The treatment devices 14 can be part of an application system, and the treatment device 14 can have various, configurations, shapes and sizes suitable for different body parts such that heat can be removed from any subcutaneous lipid-rich target area of the subject 11. For example, the treatment devices 14 may be designed to treat target areas of the patient's body, such as chin, cheeks, arms, pectoral areas, thighs, calves, buttocks, back, abdomen, “love handles” and so forth. The treatment devices 14 can have a cooling unit 15 that cools a selected area of the subject 11. As explained in more detail below, the system 10 can also include a disposable protective device and a cryoprotect for cooling the lipid-rich adipose tissue.

In the embodiment illustrated in FIG. 1, the treatment device 14 may provide mechanical energy to create a vibratory, massage and/or pulsatile effect in addition to cooling subcutaneous adipose tissue, such as the devices described in U.S. Pat. No. 7,367,341 and commonly assigned U.S. Patent Publication No. 2008/0287839. The treatment device 14, for example, may include one or more actuators that generate a transitory force which is transmitted to the subject. Suitable actuators include motors with eccentric weights, hydraulic motors, electric motors, pneumatic motors, solenoids, other mechanical motors, piezoelectric shakers and other devices that provide vibratory energy to the treatment site. A single treatment device 14 may have a plurality of different types of actuators in any desired combination. For example, the treatment device 14 may have an eccentric weight actuator (not shown) and a pneumatic motor (not shown) such that different effects may be provided with the same treatment device 14. This would provide a number of options for differential treatments of lipid rich cells within a single target area or among multiple target areas of subject 11.

The cooling unit 15 can be a component of a cooling unit integrated with the treatment device 14, and the cooling unit 15 may include one or more Peltier-type thermoelectric elements, such as a plurality of individually controlled thermal segments that create a custom spatial cooling profile and/or a time-varying cooling profile. Each custom treatment profile can include one or more segments, and each segment can include a specified duration, a target temperature, and control parameters for features such as vibration, massage, vacuum and other treatment modes. Cooling devices having multiple individually controlled heat exchanging units are described, e.g., in commonly assigned U.S. Patent Publication No. 2008/0077211.

The treatment unit 16 may be a refrigeration unit, a cooling tower, a thermoelectric chiller or cooler or any other device or cooling unit capable of removing heat from a coolant in addition to or in lieu of the cooling unit 15 at the treatment device. The treatment unit 16 can be operatively coupled to the treatment device 14 by supply and return fluid lines 18a and 18b that circulate chilled fluid (e.g., a coolant) through the treatment device 14. Alternatively, the treatment unit 16 can circulate warm fluid to the treatment device 14 during periods of warming. Examples of the circulating coolant include water, glycol, synthetic heat transfer fluid, oil, a refrigerant, a cryoprotectant and/or any other suitable heat-conducting fluid. The fluid lines 18a and 18b may be hoses or other conduits constructed from polyethylene, polyvinyl chloride, polyurethane and/or other materials that can accommodate the particular circulating coolant. Furthermore, one skilled in the art will recognize that there are a number of other cooling technologies that could be used such that the cooling units or coolers of the treatment unit 16 or the treatment device 14 need not be limited to those described herein.

The system 10 may further include a power supply 20 and a processing unit 24 operatively coupled to the treatment device 14, the cooling unit 15 and/or the treatment unit 16. In one example, the power supply 20 provides a direct current voltage to a thermoelectric element of the cooling unit 15 to adjust the heat flux over a relatively large area. The processing unit 24 may monitor process parameters via sensors (not shown) placed proximate to the treatment device 14 through power line 26 to, among other things, adjust the heat removal rate based on the process parameters. The processing unit 24 may further monitor process parameters to adjust the cooling unit 15 or other components based on the process parameters.

The processing unit 24 may be in direct electrical communication with treatment device 14 through the electrical line 22 as shown in FIG. 1; alternatively, processing unit 24 may be connected to treatment device via a wireless or an optical communication link. For example, the processing unit 24 may be in electrical communication with a control panel of the treatment device 14, the cooling unit 15 and/or an interface assembly. The processing unit 24 may be any processor, programmable logic controller, distributed control system and so on. Although the power line 26 and the electrical line 22 are shown in FIG. 1 without any support structure, these lines and other lines including, but not limited to the fluid lines 18a and 18b, may be bundled into or otherwise accompanied by a conduit or the like to protect the lines, enhance user safety and ergonomic comfort, inhibit unwanted motion that could adversely impact the heat transfer rate, provide electrical and thermal insulation and provide an aesthetic appearance to the system 10. Examples of such a conduit include a flexible polymeric fabric, a composite sheath, an adjustable arm, etc. Such a conduit (not shown) may be designed (via adjustable joints, etc.) to “set” the conduit in place for the treatment of the subject 11.

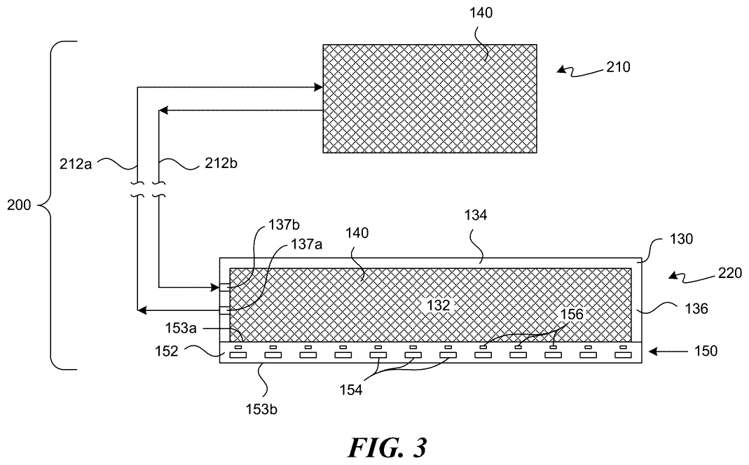

The system 10 can also include an input device 28 and an output device 30 operatively coupled to the processing unit 24. The input device 28 may be a keyboard (shown in FIG. 1), a mouse, a touch screen, a push button, a switch, a potentiometer, any combination thereof and any other device or devices suitable for accepting user input. The output device 30 may include a display or touch screen, a printer, a medium reader, an audio device, a visual device, any combination thereof and any other device or devices suitable for providing user feedback. In the embodiment of FIG. 1, the input device 28 and the output device 30 may be combined in a single unit such as a touch screen. The control panel 14b may include visual indicator devices or controls (lights, numerical displays, etc.) and/or audio indicator devices or controls. The control panel of the treatment device 14 may be a separate component from the input device and/or output device as shown in FIG. 3, or the control panel may be (a) integrated with one or more of the input and output devices 28 and 30, (b) partially integrated with one or more of the input and output devices 28 and 30, (c) at another location, and so on. In this example, the processing unit 24, the power supply 20, the treatment unit 16, the input device 28 and the output device 30 are carried by a rack or cart 34 with wheels 36 for portability. In alternative examples, the processing unit 24 may be contained in, attached to, or integrated with the treatment device 14, the cooling unit 15 and/or an interface assembly. In yet another example, the various components may be fixedly installed at a treatment site. Further details with respect to selected versions of the components and/or operation of the treatment device 14, cooling unit 15 and other components may be found in commonly-assigned U.S. Patent Publication No. 2008/0287839.

Without being bound by theory, it is believed that effective conductive cooling from the treatment device 14 depends on a number of factors. Examples of factors that impact heat removal or extraction from the skin and related tissue include, for example, the surface area of the treatment unit, the temperature of the interface member, the mechanical energy delivered to the tissue, the distribution of cryoprotectant and the extent of non-uniformities in the contact between the interface member and the skin. More specifically, upon receiving input to start a treatment protocol, the processing unit 24 can cause the treatment device 14 to cycle through each segment of a prescribed treatment plan. In so doing, the treatment device 14 applies power to one or more cooling segments, such as thermoelectric coolers (e.g., TEC “zones”), to begin a cooling cycle and, for example, activate features or modes such as vibration, massage, vacuum, etc. Using temperature or heat flux sensors, the processing unit 24 determines whether the temperature and/or heat flux at one or more areas of the actuator are sufficiently close to a target temperature or target heat flux. It will be appreciated that while a region of the body (e.g., adipose tissue) has been cooled or heated to the target temperature or by a target heat flux, in actuality that region of the body may be close but not equal to the target temperature, e.g., because of the body's natural heating and cooling variations. Thus, although the system may attempt to heat or cool to the target temperature or by a target heat flux, a sensor may measure a sufficiently close temperature. If the target temperature has not been reached, power can be increased or decreased to change heat flux, as needed, to maintain the target temperature or “set-point.” When the prescribed segment duration expires, the processing unit 24 may apply the temperature and duration indicated in the next treatment profile segment. In some embodiments, temperature can be controlled using a variable other than, or in addition to, power.

A cryoprotectant is used with the treatment device 14. Among other advantages, a cryoprotectant can assist in preventing freezing of non lipid-rich tissue (e.g., dermal tissue) during treatment. Suitable cryoprotectants and processes for implementing cryoprotectants are described in commonly-assigned U.S. Patent Publication No. 2007/0255362 and U.S. patent application Ser. No. 13/011,640 filed on Jan. 21, 2011, which are hereby incorporated by reference. As used herein, “cryoprotectant” means a flowable compound that prolongs the time to freeze non lipid-rich tissue (e.g., dermal tissue) compared to an absence of the compound.

In one example of operating the system 10, the treatment device 14 is drawn against the skin of the subject 11 to achieve efficient treatment. The subject 11 generally has a body temperature of about 37° C., which is maintained at a relatively constant level by circulation of blood. As a result, blood flow through the skin and subcutaneous layer of the region to be treated acts as a heat source that counteracts the cooling of the subdermal fat. Cooling the tissue of interest accordingly requires not only removing the heat from the target tissue but also from the blood circulating through this tissue. The efficiency of cooling the tissue can be enhanced by temporarily reducing or eliminating blood flow through the treatment region using a vacuum or other technique. Applying a vacuum may also pull skin and underlying adipose tissue away from the body which can assist in cooling underlying tissue by increasing the distance between the subcutaneous fat and the relatively well-perfused muscle tissue and by allowing the underlying adipose tissue simultaneously to be cooled from two sides.

By cooling the subcutaneous tissue to a temperature lower than 37° C., subcutaneous lipid-rich cells may be damaged selectively. In general, the epidermis and dermis of the subject 11 have lower amounts of fatty acids compared to the underlying lipid-rich cells forming the subcutaneous tissues. Because non-lipid-rich cells usually can withstand colder temperatures better than lipid-rich cells, the subcutaneous lipid-rich cells can be injured selectively while maintaining the non-lipid-rich cells in the dermis and epidermis. The presence of a cryoprotectant at and/or on the dermal tissue enhances the selectivity such that the dermal tissue can withstand even colder temperatures which induce cell death in more lipid-rich tissue. For example, the temperature range may be from about −10° C. to about 0° C.

Several embodiments of the system 10 may damage, injure, disrupt or otherwise reduce subcutaneous lipid-rich cells generally without collateral damage to non-lipid-rich cells in the treatment target area. In general, it is believed that lipid-rich cells can be affected selectively (e.g., damaged, injured or disrupted) by exposing such cells to low temperatures that do not adversely affect non-lipid-rich cells to the same extent or in the same manner. As a result, lipid-rich cells, such as subcutaneous adipose tissue, can be damaged while other cells in the same region are generally not damaged even though the non-lipid-rich cells at the surface are subject to even lower temperatures. The mechanical energy provided by the applicator may further enhance the effect on lipid-rich cells by mechanically disrupting the affected lipid-rich cells.

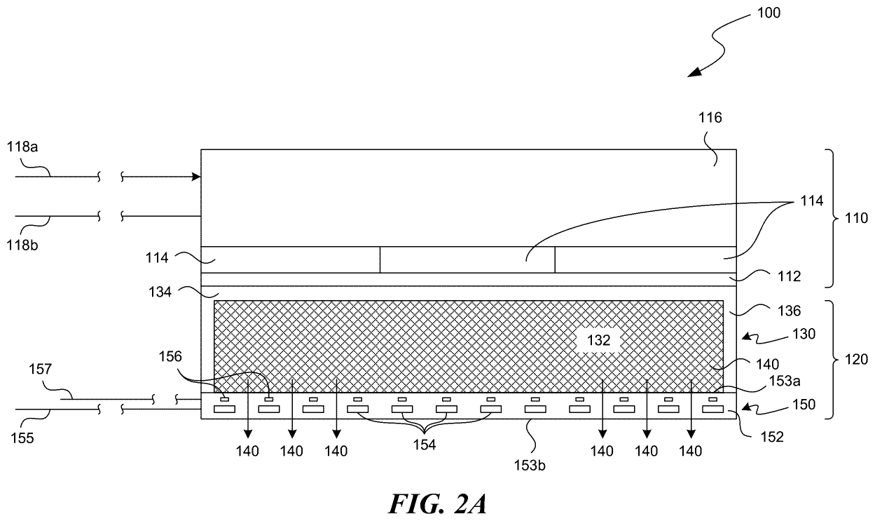

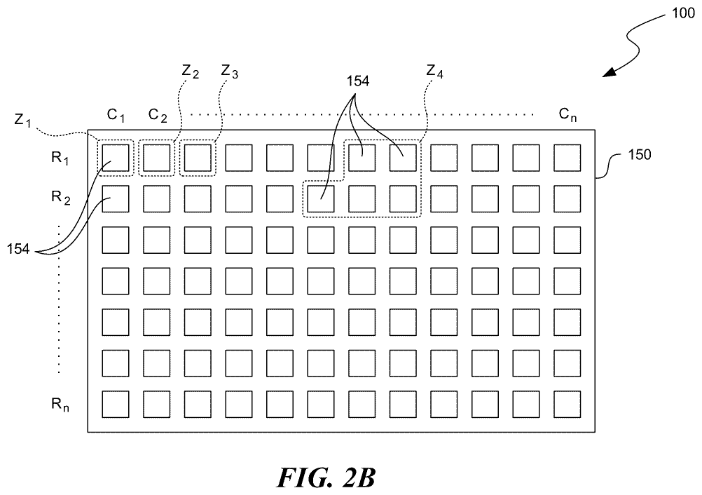

FIG. 2A is a schematic cross-sectional view of an application system 100 for cooling subcutaneous lipid-rich tissue. The application system 100, for example, may include a treatment device and a cooling unit integrated with the treatment device and/or with the treatment unit. In this embodiment, the application system 100 is a treatment device configured to contact the target area. The application system 100 can include a cooling unit 110 and an interface assembly 120 operably coupled to the cooling unit 110. The cooling unit 110, for example, may be similar to the cooling unit 15 of the treatment device 14 described above with reference to FIG. 1. The embodiment of the cooling unit 110 shown on FIG. 2A can include a plate 112 that has a high thermal conductivity, one or more Thermoelectric Elements (TEEs) 114 and a coolant chamber 116. As explained above with reference to FIG. 1, a coolant can recirculate through the coolant chamber 116 via inlet and outlet lines 118a and 118b, respectively, and the TEEs 114 can selectively heat and/or cool relative to the temperature of the coolant in the coolant chamber 116 to control the temperature over relatively large areas of the cooling plate 112. Other embodiments of the cooling unit 110 do not include the TEEs 114 such that the coolant chamber 116 extends to the cold plate 112. In either case the cooling unit 110 provides a heat sink that cools the interface assembly 120.

The interface assembly 120 further controls the heat flux through a plurality of smaller zones and delivers a cryoprotectant to the target area. In one embodiment, the interface assembly 120 includes a cryoprotectant container 130 having a cavity 132 that contains a cryoprotectant 140 and an interface element 150 through which the cryoprotectant 140 can flow. The cryoprotectant container 130 can be a rigid or flexible vessel having a back panel 134 facing the cooling unit 110 and a sidewall 136 projecting from the back panel 134. The interface element 150 can be attached to the sidewall 136 to enclose the cavity 132. The interface element 150 can include a contact member 152 having a backside 153a in contact with the cryoprotectant 140 and a front side 153b configured to contact the epidermis of the subject. The contact member 152 can be a flexible barrier (e.g., membrane) such as a porous sheet of a polymeric material or a foil with small holes, a mesh, fabric or other suitable material through which the cryoprotectant 140 can flow from the backside 153a to the front side 153b. In other embodiments, the contact member 152 can be a substantially rigid barrier that is thermally conductive and configured to allow the cryoprotectant 140 to pass from the front side 153a to the backside 153b. A rigid contact member, for example, can be a plate with holes or a panel made from a porous metal material. Suitable materials for a rigid contact member 152 include aluminum, titanium, stainless steel, or other thermally conductive materials.

The interface element 150 of the application system 100 further includes an array of heating elements 154 carried by the contact member 152. The individual heating elements 154 can be arranged in a grid or other type of pattern, and each heating element 154 is independently controlled relative to the other heating elements to provide control of the heat flux through smaller, discrete zones at the interface between the target area and the interface element 150. The heating elements 154, for example, can be micro-heaters electrically coupled to a power source via a cable 155 such that the controller can selectably address individual heating elements 154. The interface element 150 can further include a plurality of temperature sensors 156 carried by the contact member 152. The temperature sensors 156 may be arranged in an array such that one or more temperature sensors can measure the heat flux through the heat flux zones associated with one or more individual heating elements 154. The temperature sensors 156 can be electrically coupled to a control unit via a cable 157 in a manner similar to the heating elements 154.

FIG. 2B is a schematic bottom view of the interface element 150. Referring to FIGS. 2A and 2B together, the heating elements 154 can be arranged in a grid having C₁-Cₙ columns and R₁-Rₙ rows. Each individual heating element 154 can define a heat flux zone Z through which the heat flux can be selectively controlled relative to other areas of the interface element 150 (see, e.g., heat flux zones Z₁, Z₂ and Z₃). In other embodiments, a plurality of heating elements 154 can be grouped together into a set that defines a heat flux zone (see, e.g., heat flux zone Z₄). In other embodiments, the heating elements 154 can be arranged in different configurations. The temperature sensors 156 can also be arranged in the same grid as the heating elements 154. For example, each column-row address can have one or more temperature sensors 156 to measure the temperature and/or heat flux associated with each individual heating element 154 or heat flux zone.

The cable 155 (FIG. 2A) can include a plurality of individual wires that electrically couple corresponding individual heating elements 154 to a multi-channel power source. The cable 157 (FIG. 2A) can similarly include individual wires that electrically couple the temperature sensors 156 to an analog-to-digital converter, which is then coupled to a controller. In operation, the controller can operate the power source to selectively address the independent heating elements 154 based upon the column and row to provide a desired cooling profile in the subcutaneous lipid-rich tissue.

In operation, a target site of the subject is registered relative to the grid of heating elements of the interface element 150. The target site can have a single heat flux zone, or the target site can be divided into a number of different areas in which each area is associated with a corresponding heat flux zone. In either situation, a practitioner inputs the extent of desired cooling for the heat flux zones or this information can be provided to the controller by a predetermined control algorithm. The interface element 150 is positioned at the target site of the subject. In many applications, the interface element 150 and the epidermis of the target site are coapted under pressure provided by a vacuum, belt or other mechanism that forces the skin of the subject against the interface element 150. The cryoprotectant 140 in the cryoprotectant container 130 flows through the interface element 150 and contacts the skin of the subject, and a coolant is recirculated through the coolant chamber 116 of the cooling unit 110 to globally cool the back panel 134 of the cryoprotectant container 130.

The temperature at the back panel 134 can be optionally controlled regionally using TEEs 114. The cooling provided by the cooling unit 110 reduces the temperature of the cryoprotectant 140 in the cryoprotectant container 130 to a base temperature. While the interface element 150 contacts the target site, a controller selectively activates the heating elements 154 to control the heat flux through the heat flux zones across the target site. The temperature and/or heat flux at the individual heat flux zones across the target site can be monitored via the temperature sensors 156 to provide closed loop control of the heat flux according to the predetermined algorithm. Suitable algorithms for defining and controlling the heat flux are disclosed in U.S. Patent Publication No. 2010/0152824 (U.S. patent application Ser. No. 12/337,544), which is herby incorporated by reference.

Several embodiments of the application assembly 100 enhance the control of the heat flux across different regions of the target area. This can be useful because different subjects may have different deposits of lipid-rich tissue within a target area, or the subject may have particularly sensitive dermal tissue at particular regions of the target area. Moreover, the enhanced control of the heat flux through the individual heat flux zones enables more accurate control of the cooling profile within the lipid-rich tissue.

FIG. 3 is a schematic illustration of an application system 200 for cooling subcutaneous lipid-rich tissue in accordance with another embodiment of the technology. Like reference numbers refer to similar components in FIGS. 2A-3. The embodiment of the application system 200 shown in FIG. 3 includes a cooling unit 210 and treatment device having an interface assembly 220 located remotely from the cooling unit 210. For example, the cooling unit 210 can be a cooler or chiller in the treatment unit 16 located in the rack or cart 34 shown in FIG. 1. In this particular embodiment, the cryoprotectant vessel 130 further includes outlet/inlet ports 137a and 137b, respectively, and the application system 200 further includes recirculation lines 212a and 212b extending between the cooling unit 210 and the interface assembly 220. In operation, the cooling unit 210 cools and recirculates the cryoprotectant 140 through the recirculation lines 212a-b and the cavity 132 of the cryoprotectant container 130. The cryoprotectant 140 flows through the contact member 152, and the heating elements 154 control the heat flux through the heat flux zones as described above with reference to FIGS. 2A and 2B. The application system 200 of this embodiment accordingly cools the cryoprotectant remotely from the interface assembly 220. As such, the cooling unit 220 and recirculation lines 212a-b can be flushed or cleaned between treating different subjects, and/or the cryoprotectant 140 can include a germicide.

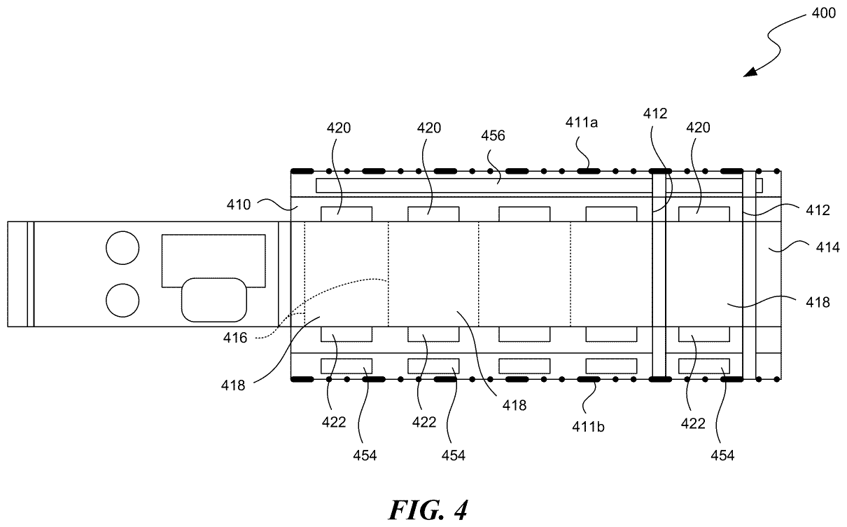

FIG. 4 is a schematic cross-sectional view of another embodiment of an interface element 400 for use in the application assembly 100 for cooling subcutaneous lipid-rich tissue. In this embodiment, the interface element 400 includes a flexible barrier 410 having a backside 411a, a front side 411b opposite the backside 411a and a plurality of channels 412 or other structure through which the cryoprotectant can pass from the backside 411a to the front side 411b (e.g., a porous structure). The interface element 400 further includes a thermal gradient layer 414 having a known thickness, thermal conductivity and heat capacity. The thermal gradient layer 414 has a plurality of striations 416 or other types of discontinuities that divide the thermal gradient layer 414 into a plurality of heat flux units 418. The interface element 400 further includes a set of temperature sensors having a dorsal temperature sensor 420 and a ventral temperature sensor 422 associated with each heat flux unit 418. The dorsal temperature sensors 420 indicate the temperature of the thermal gradient layer 414 toward the backside 411a of the barrier 410, and the ventral temperature sensors 422 indicate the temperature of the thermal gradient layer 414 toward the skin of the subject. The heat flux through each heat flux unit 418 can be determined based upon the difference between the dorsal and ventral temperature sensors 420 and 422 in combination with the known thickness, thermal conductivity and heat capacity of the thermal gradient layer 414.

The embodiment of the interface element 400 shown in FIG. 4 further includes a plurality of heating elements 454. More specifically, the interface element 400 can include one or more heating element 454 associated with individual heat flux units 418. The interface element 400 can optionally include a backside heating element 456 at the backside 411a of the barrier 410. The backside heating element 456 can globally heat the cryoprotectant at the backside 411a to provide a desired known temperature uniformly across the backside 411a. In operation, the cooling unit (not shown in FIG. 4) is set to a base temperature and the backside heating element 456 is adjusted to provide the desired backside temperature of the cryoprotectant. This enables quick and accurate adjustment of the set point for the cryoprotectant entering the interface element 400. The heating elements 454 are then controlled to provide the desired heat flux through the individual heat flux units 418. As described above, a heat flux zone can be defined by a single heat flux unit 418, or a set of heat flux units 418 can define a heat flux zone.

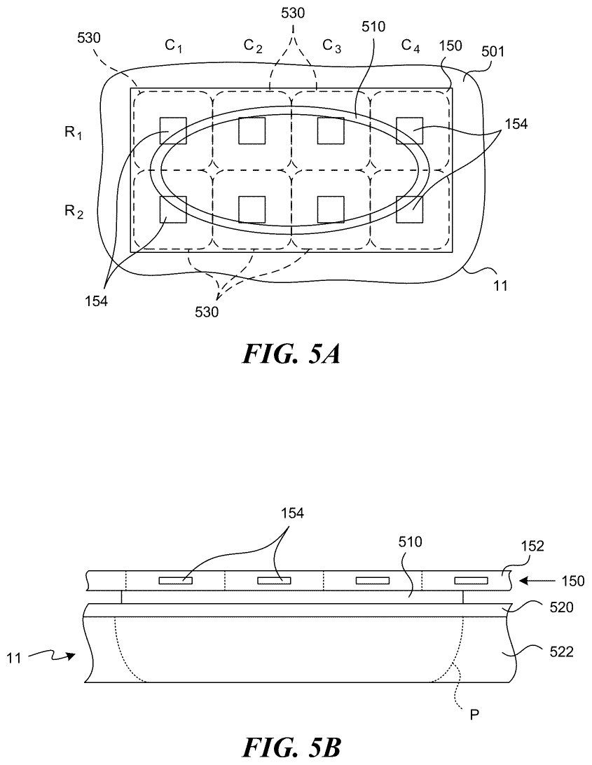

FIGS. 5A and 5B illustrate another implementation of application assemblies for cooling subcutaneous lipid-rich tissue in accordance with the technology. In this embodiment, one or more markers 510 are placed on the skin 520 of the subject 11 such that the markers 510 define the perimeter of the target area. The interface assembly 120 is positioned so that a number of the heating elements 154 are superimposed over the region defined by the markers 510. The heating elements 154 are then controlled based upon the outline of the target area defined by the markers 510 and the desired heat flux through the various heat flux zones 530. In the illustrated embodiment, for example, the heat flux through the heat flux zones 530 associated with columns C₂ and C₃ can be greater than that through the corresponding heat flux zones 530 associated with columns C₁ and C₄. This can be achieved by providing more heat to the heating elements in columns C₁ and C₄ compared to those in columns C₂ and C₃ and/or the smaller surface areas defined by the markers 510 in columns C₁ and C₄. Referring to FIG. 5B, this provides a controlled treatment profile P that extends through the skin 520 and into the lipid-rich tissue 522 for reducing the volume of the lipid-rich tissue as described above.

The markers 510 can be conductive members or dielectric templates. In one example, the markers 510 are a conductive ink or magnetic ink that is deposited on the skin 520 of the patient around the perimeter of the treatment area. The heating elements 154 can include sensors that detect the presence of the inks, and the controller can then selectively operate the heating elements based on the outline of the markers 510. In another example, the markers 510 are defined by a dielectric template that has an opening in the shape of the treatment area. The heating elements 154 can be operated to focus the heat flux through the opening of the template. For example, the heating elements 154 can sense the heat flux through the corresponding heat flux zones, and the controller can determine the shape of the opening based on a greater heat flux through the opening compared to areas covered by the dielectric material.

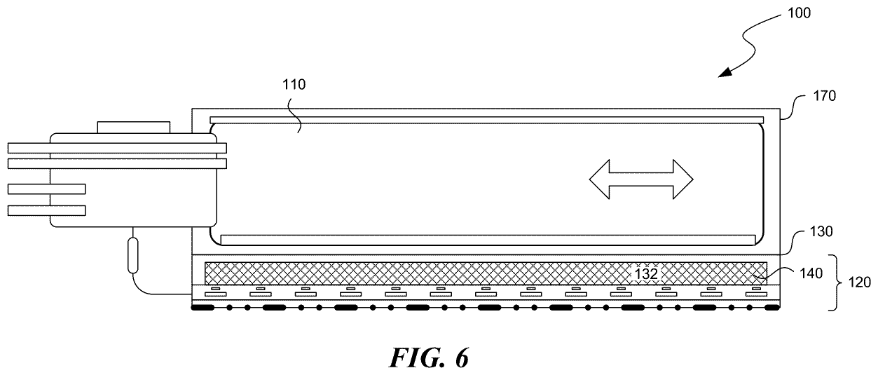

FIG. 6 is a schematic cross-sectional view of another embodiment of the application system. In this embodiment, the interface assembly 120 further includes a connector 170 that couples the cryoprotectant vessel 130 to the cooling unit 110. The connector 170 can comprise a sheath or pocket in which cooling unit 110 is received to treat a subject. After performing the treatment, the interface assembly 120 can be removed and disposed appropriately. The cooling unit 110 can then be inserted into a new, sterile interface assembly for treating the next subject.

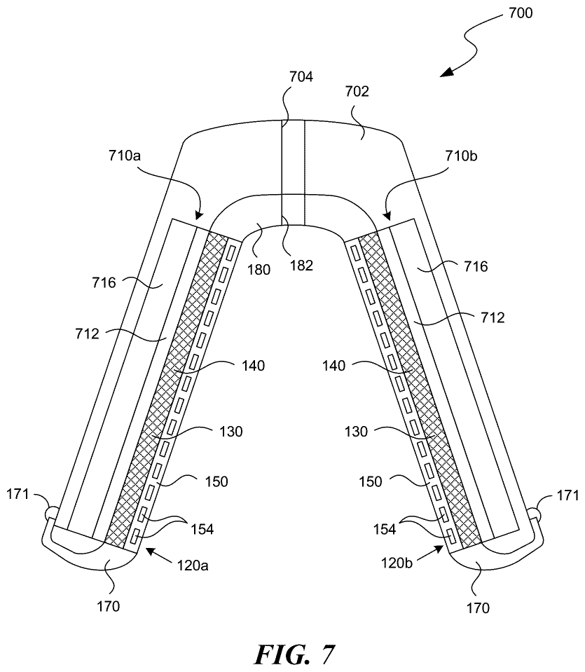

FIG. 7 is a schematic cross-sectional view of an application system 700 for cooling lipid-rich tissue in accordance with another embodiment of the technology. The treatment device 700 includes a vacuum cup 702 having a vacuum port 704, a first cooling unit 710a on one side of the cup 702, and a second cooling unit 710b on an opposing side of the cup 702. Each of the first and second cooling units 710a and 710b can be similar to the cooling unit 110 described above with reference to FIGS. 2A and 2B. As such, each of the cooling units 710a-b can include a cold plate 712 and a coolant chamber 716. The cooling units 710a-b can be fixed to the vacuum cup 702.

In this embodiment, the treatment device 700 also includes a first interface assembly 120a and a second interface assembly 120b within the vacuum cup 702. More specifically, the first interface assembly 120a is adjacent the first cooling unit 710a and the second interface assembly 120b is adjacent the second cooling unit 710b. The first and second interface assemblies 120a-b can further include connectors 170 in the form of tabs or other mechanisms that can be secured by clasps or clamps 171 to releasably attach the first and second interface assemblies 120a-b to the vacuum cup 702. The first and second interface assemblies 120a-b can also be connected to each other by a flexible intermediate portion 180. The intermediate portion 180 can include a vacuum port 182 aligned with the vacuum port 704 of the vacuum cup 702, or the intermediate portion 180 can have a porous through which air can flow. In operation, the rim of the vacuum cup is placed against the skin of a subject and a vacuum is drawn within the cup. The vacuum pulls the tissue of the subject into the cup 702 and coapts the target area with the interface elements 150 of the corresponding first and second interface assemblies 120a-b. One suitable vacuum cup 702 with cooling units is described in U.S. Provisional Patent Application Ser. No. 61/174,407, filed on Apr. 30, 2009, and incorporative by reference above.

From the foregoing, it will be appreciated that specific embodiments of the invention have been described herein for purposes of illustration, but that various modifications may be made without deviating from the spirit and scope of the invention. The application systems, interface assemblies and methods may be combined in further embodiments. For example, the interface element 400 shown and described with reference to FIG. 4 can be used in any of the interface assemblies 120, 120a-b or 200 described above with reference to FIGS. 2A-3 and 5A-7. In addition, while advantages associated with certain embodiments have been described and the context of those embodiments, other embodiments may also exhibit such advantages. Not all embodiments need necessarily exhibit such advantages to fall within the scope of the present disclosure. Accordingly, the invention is not limited except as by the appended claims.

1. An application system for cooling subcutaneous lipid-rich tissue, comprising:
a cooling unit;
a cryoprotectant vessel configured to contain a fluidic cryoprotectant such that at least a portion of the cryoprotectant is cooled by the cooling unit to a desired cold temperature;
a contact member configured to be attached to the cryoprotectant vessel, the contact member including a backside configured to contact the cryoprotectant and a front side opposite the backside, and wherein the contact member is configured to allow the cryoprotectant to flow from the backside to the front side; and
an array of selectably addressable heating elements carried by the contact member.

2. The application system of claim 1, wherein:
the cooling unit comprises a heat exchanger having a coolant chamber through which a coolant can flow;
the cryoprotectant vessel comprises a backpanel and a sidewall projecting from the backpanel, the contact member is a flexible barrier attached to the sidewall of the cryoprotectant vessel to form a cryoprotectant chamber, the flexible barrier and the cryoprotectant vessel together form a disposable interface assembly, and the interface assembly further comprises a connector coupling the cryoprotectant vessel to the cooling unit;
the application system further comprises an array of temperature sensor sets carried by the flexible barrier, wherein individual heating elements are associated with a corresponding temperature sensor set;
the application system further comprises a large heating element spaced apart from the array of temperature sensor sets; and
the application system further comprises a controller including a computer-operable medium programmed to receive sensed temperatures from the temperature sensor sets and adjust the associated heating elements based on the sensed temperatures and a desired heating profile, thereby providing localized temperature differentials in the cryoprotectant corresponding to the desired heating profile.

3. The application system of claim 1, wherein the cooling unit comprises a cold plate and a heat exchanger having a coolant chamber through which a coolant can flow, and wherein the heat exchanger is in thermal communication with the cold plate.

4. The application system of claim 3, wherein the cooling unit further comprises at least one thermoelectric element between the cold plate and the heat exchanger.

5. The application system of claim 1, wherein the contact member is a flexible barrier attached to the cryoprotectant vessel such that the flexible barrier and the cryoprotectant vessel together form a disposable interface element.

6. The application system of claim 5, wherein the interface element further comprises a connector coupling the cryoprotectant vessel to the cooling unit.

7. The application system of claim 6, wherein the connector comprises a sheath associated with the cryoprotectant vessel, and the cooling unit is received within the sheath.

8. The application system of claim 7 wherein the sheath and the cryoprotectant vessel are integrally formed together such that the sheath extends from the cryoprotectant vessel.

9. The application system of claim 6 wherein the connector comprises a tab extending from the cryoprotectant vessel, and wherein the application assembly includes a clasp configured to releasably hold the tab.

10. The application system of claim 1, wherein the cryoprotectant vessel comprises a backpanel facing the cooling unit and a sidewall projecting from the backpanel, and wherein the contact member is a flexible barrier attached to the sidewall of the cryoprotectant vessel to form a cryoprotectant chamber.

11. The application system of claim 10, wherein the interface assembly further comprises a pad containing a cryoprotectant gel in the cryoprotectant chamber.

12. The application system of claim 10, wherein the cryoprotectant vessel further comprises an inlet into the cryoprotectant chamber and an outlet from the cryoprotectant chamber such that a liquid cryoprotectant can flow through the cryoprotectant chamber.

13. The application system of claim 1, wherein the cryoprotectant vessel further comprises an inlet and an outlet configured to direct a flow of a liquid cryoprotectant through the cryoprotectant vessel.

14. The application system of claim 13, wherein:
the cryoprotectant vessel comprises a backpanel, a sidewall projecting from the backpanel, an inlet and an outlet;
the contact member is a flexible barrier attached to the sidewall of the cryoprotectant vessel to form a cryoprotectant chamber, wherein the inlet and outlet of the cryoprotectant vessel are configured to direct a flow of liquid cryoprotectant through the cryoprotectant chamber; and
the cooling unit comprises a cold plate and a heat exchanger having a coolant chamber through which a coolant can flow, and wherein the cold plate is in contact with the backpanel of the cryoprotectant vessel.

15. The application system of claim 13, wherein:
the cryoprotectant vessel comprises a backpanel, a sidewall projecting from the backpanel, an inlet, an outlet, and a conduit having one portion connected to the inlet and another portion connected to the outlet;
the contact member is a flexible barrier attached to the sidewall of the cryoprotectant vessel to form a cryoprotectant chamber, wherein the inlet and the outlet of the cryoprotectant vessel are configured to direct a flow of liquid cryoprotectant through the cryoprotectant chamber; and
the cooling unit is spaced apart from the cryoprotectant chamber and coupled to the conduit, and wherein the cooling unit cools the flow of liquid cryoprotectant.

16. The application system of claim 1, wherein the array of heating elements is arranged in a grid, and individual heating elements can be independently controlled from each other.

17. The application system of claim 16, further comprising an array of temperature sensors carried by the contact member, wherein individual heating elements are associated with at least one corresponding temperature sensor.

18. The application system of claim 17, further comprising an array of temperature sensor sets carried by the contact member, wherein individual heating elements are associated with a corresponding temperature sensor set.

19. The application system of claim 1, further comprising a large heating element spaced apart from the array of heating elements.

20. An application assembly for cooling subcutaneous lipid-rich tissue, comprising:
a cooling unit; and
a disposable interface assembly having a connector configured to retain the disposable interface assembly in contact with the cooling unit, a cryoprotectant vessel proximate the cooling unit such that a cryoprotectant is cooled to a desired base temperature, and an interface element having a contact member through which the cryoprotectant can pass and an array of addressable heating elements carried by the flexible contact member.

21.-25. (canceled)
